(19) United States
(12) Patent Application Publication
(10) Pub. No.: US 2025/0299769 A1
(43) Pub. Date: Sep. 25, 2025

(54) MULTIOMIC DATA INTEGRATION WITH MACHINE LEARNING AND MODEL INTERPRETATION

(71) Applicant: The Medical College of Wisconsin, Inc., Milwaukee, WI (US)

(72) Inventors: Jesse Gerard Meyer, Milwaukee, WI (US); Quinn Peter Dickinson, New Berlin, WI (US)

(21) Appl. No.: 18/863,931

(22) PCT Filed: May 10, 2023
(86) PCT No.: PCT/US2023/021738

§ 371 Date: Nov. 7, 2024

Related U.S. Application Data
(60) Provisional application No. 63/340,356, filed on May 10, 2022.

(51) Int. Cl. G16B 5/00; G06F 30/27; G16B 40/20

(52) U.S. Cl. CPC G16B 5/00; G06F 30/27; G16B 40/20

(57) ABSTRACT

Multiomics integration analysis is provided using machine learning and model interpretation. Feature data that indicate connections between different layers of a multiomics dataset are generated. Based on these feature data, connections between a first type of omics data (e.g., proteomics data) and a second type of omics data can be determined. One or more machine learning algorithms or models are used to generate output data, from which model interpretation data are generated, and based on which feature data that indicate interactions between biomolecules across layers of omics data are generated.

### CROSS-REFERENCE TO RELATED APPLICATIONS

This application claims the benefit of U.S. Provisional Patent Application Ser. No. 63/340,356, filed on May 10, 2022, and entitled “MULTIOMIC DATA INTEGRATION WITH MACHINE LEARNING AND MODEL INTERPRETATION,” which is herein incorporated by reference in its entirety.

### STATEMENT OF FEDERALLY SPONSORED RESEARCH

This invention was made with government support under GM142502 and AG074234 awarded by the National Institutes of Health. The government has certain rights in the invention.

### BACKGROUND

Cells respond to environments by regulating gene expression to optimally exploit resources. Recent advances in technologies allow for measuring the abundances of RNA, proteins, lipids, and metabolites. These highly complex datasets reflect the states of the different layers in a biological system. Multiomics is the integration of these and other disparate omics methods and data (e.g., genomics, epigenomics, microbiomics, lipidomics, and so on) to gain a clearer picture of the biological state. Multiomic studies of the proteome and metabolome or other aspects of a biological state or system are becoming more common as mass spectrometry and other measurement technologies continue to be democratized. However, knowledge extraction through integration of these data remains challenging.

There are various methods to integrate multiomic datasets. Multiomic integration strategies are currently employed within three general disciplines: (1) disease subtyping, especially in the context of cancer heterogeneity; (2) biomarker discovery; and (3) discovery of biological insights.

In the context of biological insights, multiomics integration has been accomplished using several statistical approaches, such as Bayesian or correlation-based approaches. These approaches have uncovered pathways involved in cancer prognosis, drug selectivity of cancer lines, and novel candidate oncogenes. However, most existing multiomic data integration methods are not able to infer new biological interactions between layers of multiomic data, and the methods that do look for connections between layers often look at 1:1 connections based on simple linear correlation. Due to complex biological regulation balancing many processes, many interesting connections between omic layers are unlikely to have 1:1 relationships. There is a need for new strategies that leverage the interactions between omics layers to discover non-linear relationships and produce more knowledge than the sum of the two datasets.

Machine learning is a promising approach for discovering relationships between datasets. Machine learning techniques have found success in the integration of multiomic datasets for particular prediction tasks. Some examples of this include supervised methods predicting cancer prognosis, cellular state in E. coli, patient survival outcomes for cancer types, or patient drug response. Unsupervised methods have also been developed for the discovery of biomarkers and the subtyping of cancers. Each of these approaches rely on an early, intermediate, or late integration strategy. The integration of multiomic data through hierarchical prediction between omic layers is relatively unexplored.

### SUMMARY OF THE DISCLOSURE

The present disclosure addresses the aforementioned drawbacks by providing a method for generating feature data indicative of an integration between different layers of multiomics data, where each individual layer of the multiomics data can include, but not be limited to, genomics data, epigenomics data, transcriptomic data, proteomic data, metabolomic data, and so on. A first omics dataset is accessed with a computer system, where the first omics dataset comprises a first omics data type. A machine learning model is also accessed with the computer system, where the machine learning model has been trained on training data to predict a second omics data type from the first omics data type. As a non-limiting example, the first and second omics data types can be proteomics and metabolomics data. The first omics dataset is input to the machine learning model via the computer system, generating output data as predictive values of a second omics dataset comprising the second omics data type. Model interpretation data are then generated from at least one of the machine learning model, the first omics data, or the output data, where the model interpretation data indicate features in the multiomics dataset that are predictive of connections between the first omics dataset and the second omics dataset. Feature data can then be generated with the computer system based on the model interpretation data, where the feature data indicate connections between the first omics dataset and the second omics dataset.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 is a flowchart illustrating the steps of an example method for implementing a multiomics integration method (“MIM”) according to some embodiments described in the present disclosure.

FIGS. 2A-2F. MIMaL workflow, model interpretation, and demonstration of biological applicability. (A) MIMaL is a multiomic integration method utilizing machine learning model interpretation with cluster analysis to uncover unknown relationships between samples. (B) Comparison of the model performance with average mean squared error across five folds from 5-fold cross validation. ExtraTrees (i.e., Extremely Randomized Trees) was selected for further analysis due to performance and specialized interpretation algorithms for decision tree-based methods. (C) Performance of Extra Trees models in predicting fold change in each metabolite from proteomic data, measured by R2 between predicted and experimental metabolite values for each held out test set. (D) Example of true versus predicted quantity of one metabolite, citric acid, with each point representing one sample, i.e. knockout strain under fermentation or respiration conditions. (E) SHAP forceplot for MEF1delta under respiration conditions where red and blue bars represent protein quantities that increase or decrease the prediction value of citric acid relative to the baseline, respectively. (F) Quantification of citric acid in strains selected from SHAP analysis. Strains were grown under respiration conditions, metabolites were extracted in methanol, and citric acid quantities were measured using targeted MS/MS. Citrate quantities reflect predictions made from SHAP.

FIG. 3. Correlated proteins to top 20 SHAP values. The proteins represented by the SHAP values with the greatest average magnitude were selected for further analysis by determining their correlated proteins, to better explain the selection of these protein quantities as markers for the prediction of citric acid through the ExtraTrees model. Proteins were grouped through their shared correlations and SHAP value. Groups were examined through GO term enrichment analysis of biological processes. Each group was labeled by a summary reflective of the other terms.

FIGS. 4A-4G. MIMaL clustering, interpretation, and validation. (A) Overview of the method to find connections between conditions using dimensionality reduction, clustering, and network analysis. SHAP values were calculated for all proteins across all knockouts. UMAP was used to reduce dimensionality to 10 dimensions, the first two are displayed graphically. UMAP dimensions were clustered with OPTICS. UMAP and OPTICS were repeated 1000 times for each metabolite. (B) A graph was constructed where each edge is linearly proportional to the count of co-clustering across the 69,000 clustering repetitions and setting a cutoff for edges. (C) Autoradiographic image of gel assessing mitochondrial translation in wild type and YDL157CΔ cells were treated with cycloheximide and using 35S-methionine for 15 minutes (pulse) at 30° C. The labeling was stopped by adding excess cold methionine and the temperature was increased to 37° C. to induce protein destabilization (chase for a total of 90 min). (D) Resistance to canavanine stress. Strains were grown on synthetic complete media minus arg+2.5 μg/ml canavanine for 18 days. ISC1 and SDH9 knockout strains were connected to PIL1, which was previously shown to resist canavanine stress like CAN1 (positive control). Both ISC1 and SDH9 showed resistance to canavanine compared to wild type. (E) Oxygen consumption in responses to succinate as the sole carbon source measured by seahorse respirometry. Responses to succinate were significantly different (p-value-0.001, Tukey's HSD) between SDH1 and SDH9 knockouts. (F) The strains FMP40 and FMP52 were tested for resistance to hydrogen peroxide stress under respiration (F) and fermentation (G) conditions and compared using image analysis of drop dilution assays (FIG. 7G). Differences between all strains was significant (p-value=0.001, Tukey's HSD) under fermentation conditions, and significant (p-value=0.001, Tukey's HSD) between wild-type and the others under respiration conditions.

FIGS. 5A-5E. Correlations and SHAP Values as Measures of ProC Over Citric Acid. (A) Mean absolute SHAP values for each protein are plotted with each protein's Spearman correlation with citric acid. The top ten magnitude SHAP proteins are labeled. (B) Ranking of proteins by mean absolute SHAP values and absolute Spearman correlations are compared. Although AAT2 is both the top rank for SHAP and correlation, the other top ranked SHAP proteins lay considerably lower, illustrating that the discovery of ProC of metabolites through SHAP discovers relationships beyond simple correlations. (C) Correlation of citric acid and protein values with members of the top ProC for citric acid. The usage of a linear correlation between proteins and a metabolite as a ProC can be inaccurate due to examples where the correlation is false, highlighted in red. (D) Correlation of SHAP values with citrate levels. Higher correlations are seen between SHAP and citrate than looking at direct protein folds. (E) Correlation of protein values with SHAP contributions of members of the top ProC for citric acid. While correlated, the SHAP values show a non-linear relationship with protein concentrations at the outliers, adjusting the relationship to better predict citrate.

FIGS. 6A-6D. MIMaL reveals new connections between proteins and metabolites. (A) Top 10 SHAP values for citric acid across all conditions, sorted by mean magnitude SHAP by each protein. (B) A network consisting of the metabolic pathways present in S. cerevisiae was constructed from data obtained from Biocyc. Connections between proteins through metabolites have a weight of 6. Positive genetic interactions among all ORFs in yeast were downloaded from Saccharomyces Genome Database and added to the network with a weight of 10. Distance to citrate was calculated using the Dijkstra algorithm, and can be represented by 3+(#genetic interactions)*10+(#metabolic reactions)*6. (C) The overall distribution of distances were plotted as a histogram. The network was organized by distance to citrate and the proteins representing the top 10 SHAP values for citric acid prediction were highlighted, along with their paths to citric acid. (D) A representation of the total number of nodes and connections between each category.

FIGS. 7A-7C. Additional Evidence Supporting Mitochondrial Translation MIM connections. Wild type and YJR120WΔ (A) or YJR120WΔ (B) cells were treated with cycloheximide and mitochondrial translation products were labeled with 35S-methionine for 15 minutes (pulse) at 30° C. The labeling was stopped by adding excess cold methionine and the temperature was increased to 37° C. to induce protein destabilization (chase for a total of 90 min). Loading controls are included. Differences in translation between the strains reflect the connection between YJR120W and the mitochondrial ribosome genes such as MRP1. (C) Each strain was tested for their ability to grow under respiration conditions by comparing spotting on YPD and YPG.

FIGS. 8A-8C. Additional Evidence Supporting Eisosomal MIM connections. (A) Growth of strains related to PIL1 on SC−arg. (B) Resistance to canavanine was tested in strains related to PIL1 by exposure to high concentrations of canavanine over 72 hours. Growth of each strain was tested before and after exposure and quantified using imagej. Significant differences (p-value <=0.024, Tukey's HSD) in growth were seen between wild-type and all other strains, except PIL1. (C) Responses to ethanol were compared in SDH1 and SDH9. Responses to ethanol were significantly different (p-value=0.019, Tukey's HSD) between SDH1 and SDH9.

FIGS. 9A-9B. Additional Evidence Supporting Oxidative Stress Response MIM connections. (A) The strains FMP40 and FMP52 were tested for resistance to hydrogen peroxide stress under fermentation and respiration conditions. Resistance was quantified by calculating the ratio of growth before hydrogen peroxide treatment to growth after hydrogen peroxide treatment. Growth was quantified by measuring the greyscale of a circular area from the center of each drop. (B) Resistance to hydrogen peroxide was assessed by a zone of inhibition assay on YPG for FMP40 and FMP52. Growth closer to the source of hydrogen peroxide is seen in both FMP40 and FMP52 when compared to wild type.

FIGS. 10A-10C. MIM Recapitulates Unique Set of Known Connections. (A) From the set of all connections between knockouts across all metabolites, the most important were selected by calculating a linear regression between the weight and the rank of the value. All connections with a weight above 8210 were kept for further analysis. (B) Weights calculated from MIM were compared with proteome-proteome correlations from the Y3K dataset for both respiration and fermentation. (C) Connections between knockouts were compared with correlations between knockouts in identifying known relationships between genes. A set of all known positive and negative genetic interactions and physical interactions was compared with the set of connections and correlations. A unique set of interactions was recapitulated from both MIM and correlations.

FIG. 11 is a block diagram of an example multiomics integration method (“MIM”) system.

FIG. 12 is a block diagram of example components that can implement the system of FIG. 11.

FIGS. 13A-13E. Example graphical user interface for easy and fast data exploration with MIMaL. (A) The “SHAP Summary” page was used to see that the model effectively learned to predict the quantity of biotin from the proteome, and (B) the same page showed that RKI1 protein was the most important predictor of biotin across all conditions, followed by ARO9 and ARI1. (C) The same page also shows a plot of each protein's correlation with that metabolite (x axis) versus the mean absolute SHAP of that protein's control over the metabolite. This can show where a metabolite is not correlated positively or negatively but has high model importance. One such example for biotin is marked, DUR12. (D) The “Correlation” page was used to check how SHAP values for DUR12's control over biotin related to the quantity of biotin revealing a complex relationship. The same page also can show that DUR12 protein does not correlate with biotin (not shown). (E) Unrelated to the example in A-C, the “Network” page can be used to explore how the genes were related based on summarizing the protein control values. This network matches the network shown in FIG. 4. This enables user interaction with the network so that readers can make their own hypothesis about gene relationships. When a gene in the main network is clicked, its immediate connections show in the adjacent mini network diagram.

### DETAILED DESCRIPTION

Described here are systems and methods for multiomics integration analysis, in which feature data that indicate connections between different layers of a multiomics dataset are generated. Based on these feature data, connections between a first type of omics data (e.g., proteomics data) and a second type of omics data can be determined. Advantageously, the disclosed systems and methods allow for extracting feature data or otherwise computing predictions and generating insights from multiple omic datasets (e.g., proteomics data, metabolomics data, genomics data). Thus, in general, one or more machine learning algorithms or models are used to generate feature data that indicate, or to otherwise determine, interactions between biomolecules across layers of omics data.

As a non-limiting example, a machine learning model is trained on training data to predict one omic layer from an input of another omic layer. For instance, the model can be trained to predict metabolomic data from an input of proteomic data, to predict proteomic data from an input of metabolomic data, or to more generally predict a first type of omics data by inputting a second type of omics data into the machine learning model. The model can then be interrogated to output feature data indicating which input molecules were most relevant for predicting specific output molecules. Advantageously, the disclosed systems and methods can discover connections between proteins and metabolites. As another example, this framework can be implemented to discover connections between mRNA and proteins, or generally between any two omic layers. Advantageously, the systems and methods can thus be used to discover or otherwise investigate what regulates a drug target. For example, if a protein causes disease, the systems and methods described in the present disclosure can generate feature data, or otherwise estimate predictions indicating which metabolite or metabolites regulate that protein. A drug can then be tailored to mimic that metabolite, or metabolites.

In some embodiments, connections between omic layers can be discovered through a combination of machine learning and model interpretation. As a non-limiting example, model interpretation data, such as shapely additive explanations (“SHAP”) value data connecting different omics data types (e.g., connecting proteins to metabolites) can be used to generate feature data that indicate specific connections between different layers in a multiomics dataset that may not be identifiable with correlation-based analyses alone. In general SHAP values assign each feature an importance value describing how each of the model inputs lead to a particular prediction. In this way, SHAP value data not only indicates those features that are more relevant for a particular predictive outcome, but also indicate which values of those features are more or less likely to drive the particular predictive outcome. Advantageously, SHAP values interpret each example input separately in comparison to other methods that usually compute feature importance for the whole dataset. Additionally or alternatively, other model interpretation data can also be generated and used, such as local interpretable model-agnostic explanation (“LIME”) value data.

As a non-limiting example, clustering the magnitudes of protein control (“ProC”) values over a set of metabolites can enable the prediction of gene functions. For instance, in an example study, two uncharacterized genes in yeast were predicted to modulate mitochondrial translation: yjr120w and yld157c. As another example, functions for several incompletely characterized genes were predicted and validated, including SDH9, ISC1, and FMP52. As will be described in more detail below, the disclosed systems and methods demonstrate that multiomic analysis with machine learning (“MIMaL”) is a framework that can reveal new insight from multiomic data that would not be possible using any omic layer alone.

As noted above, over the past decade large-scale utilization of omics technology has grown, including a trend toward more studies describing combined measures of more than one omic layer, so called “multiomics.” However, few computational data integration methods, if any, take advantage of the relationships between multiple omic layers. The systems and methods described in the present disclosure present a solution to these problems and enable new insights into basic biology from multiomic datasets, thereby enabling progress in drug discovery that would otherwise be obscured by lack of holistic models of biological systems.

The systems and methods described in the present disclosure fill this gap with a multiomic data integration framework that uses machine learning. As a non-limiting example, one omic layer can be used as an input to effectively predict another omic layer (e.g., proteomic data can predict metabolomic data), and that analysis of the learned model can reveal new connections between omic layers. These connections from machine learning model interpretation are different from those revealed by protein/metabolite correlation, providing a unique insight into multiomic data analysis not previously attainable.

Model interpretation can lead to measures of how members of one omic layer or data type control members of a second omic layer or data type, and this control data can be used to reveal new biological functions. For instance, the protein control values derived from the model analysis framework described in the present disclosure can be summarized to reveal new gene functions, as mentioned above.

As noted above, the systems and methods described in the present disclosure can be used to generate feature data that indicate connections between different layers (or types) of multiomics data. As one non-limiting example, these new connections can be used to discover relationships between biological conditions when the source of the multiomics data includes two or more omics layers that have been acquired from the same sample. The connections that are discovered using the systems and methods described in the present disclosure are indicative of the measures of control that one omics layer has over another omics layer. If those measures of control are summarized across all input conditions with dimension reduction methods, such as uniform manifold projection and approximation (“UMAP”), then a similarity between conditions can be determined based on co-clustering of points that represent the input conditions in a dimension reduced space. Algorithms for determining clusters such as OPTICS can be used to determine neighbors. In one embodiment, when the biological conditions are defined as single gene knockouts, and the input data to the machine learning model therefore reflects changes in the system resulting from loss of one gene, the relations discovered from summarizing the profiles of how one omic layer exerts control over another layer can be used to infer gene function.

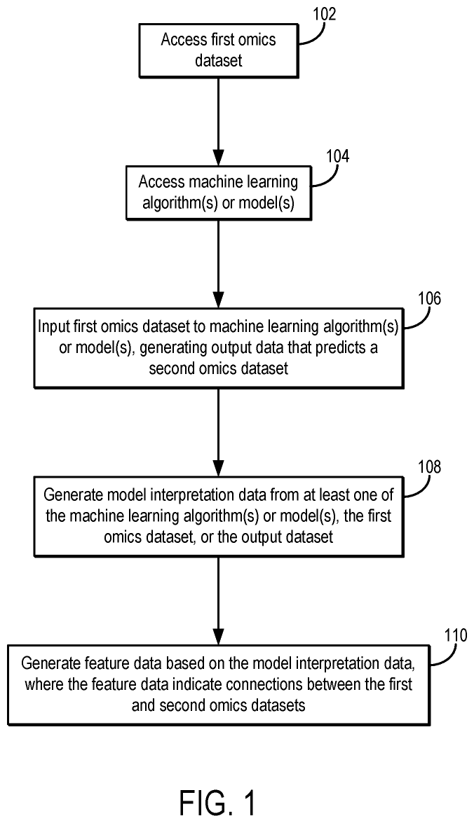

Referring now to FIG. 1, an example method for multiomics integration is illustrated. The method includes accessing a first omics dataset with a computer system, as indicated at step 102. The first omics dataset corresponds to a first omics data type. As a non-limiting example, the first omics data type can include one of proteomics data, metabolomics data, genomics data, epigenomics data, transcriptomics data, lipidomics data, and so on.

A machine learning model is also accessed with the computer system, as indicated at step 104. In general, the machine learning model has been trained on training data to predict a second omics data type from an omics dataset corresponding to the first omics data type. For example, the machine learning model can be trained to predict metabolite changes from proteomic changes (e.g., by predicting metabolomics data from an input of proteomics data).

The first omics dataset is input to the machine learning model, generating output data, as indicated at step 106. For instance, the output data can include predicted or otherwise estimated values of a second omics dataset corresponding to a second omics data type. As discussed above, it is an advantage of the present disclosure that model interpretation data can additionally be generated and analyzed to generate feature data that better represent the predictive connections between multiomics data layers. Thus, as indicated at step 108, model interpretation data are generated from the machine learning algorithm(s) or model(s), the input data, and/or the output data. In general, the model interpretation data can indicate connections between the first omics dataset and the output data (i.e., an omics dataset corresponding to a second omics data type). As a non-limiting example, the model interpretation data can include a set of features in the first and/or second omics datasets; rankings of some or all of those features in terms of how relevant they are to the predictive connections between different multiomics data layers; quantitative values associated with each of the features; and/or measures of which values of features have positive or negative predictive effects. As a non-limiting example, the model interpretation data can include SHAP values.

Based at least in part on the model interpretation data, feature data are generated with the computer system as indicated at step 110. For instance, the feature data can indicate connections between the first omics dataset and the second omics dataset. It is an advantage of the disclosed systems and methods that the feature data can indicate connections between different layers of multiomics data that are otherwise not identifiable based on correlation analyses alone. As an example, the feature data can indicate connections between the input conditions, and when the input conditions are single gene knockouts this can suggest function of the gene. The feature data can be displayed to a user, or stored for later use (e.g., additional analyses on the multiomics data).

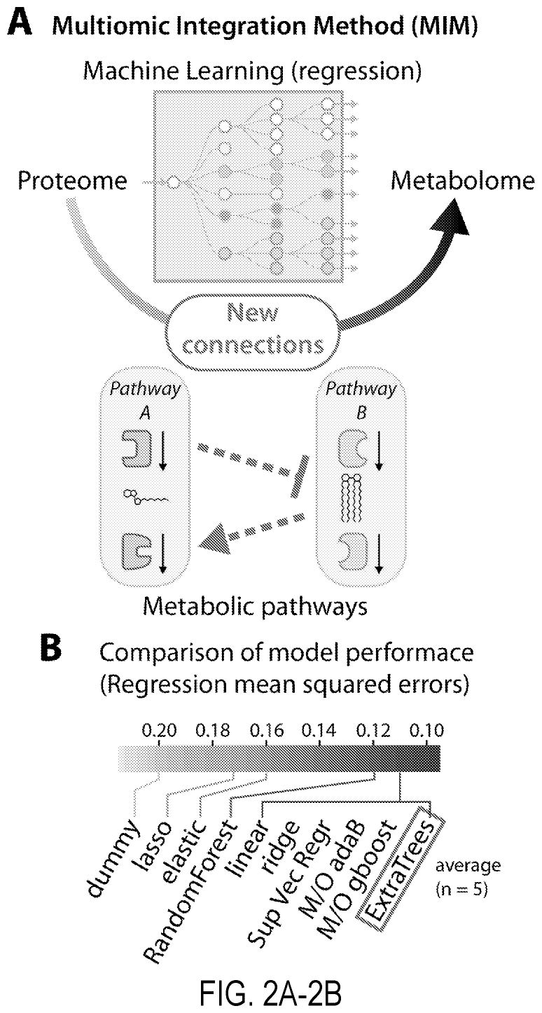

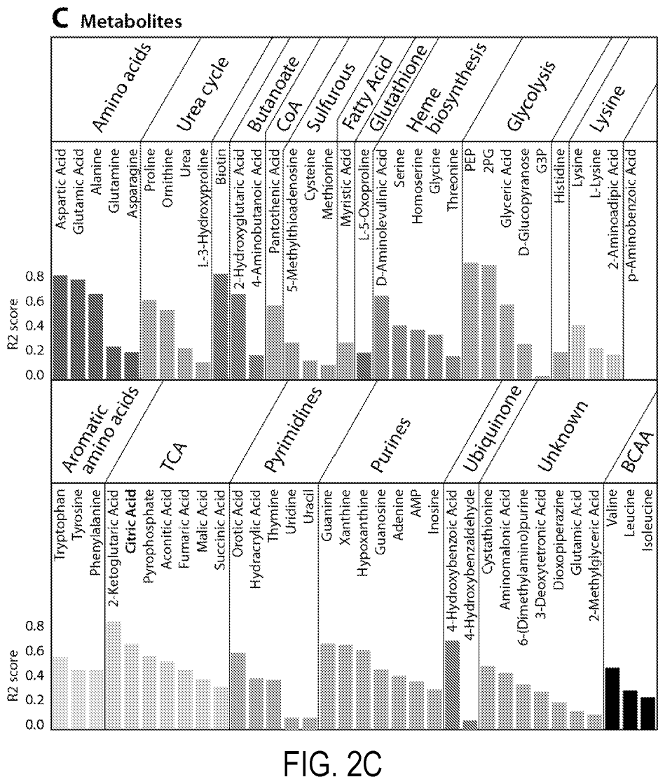

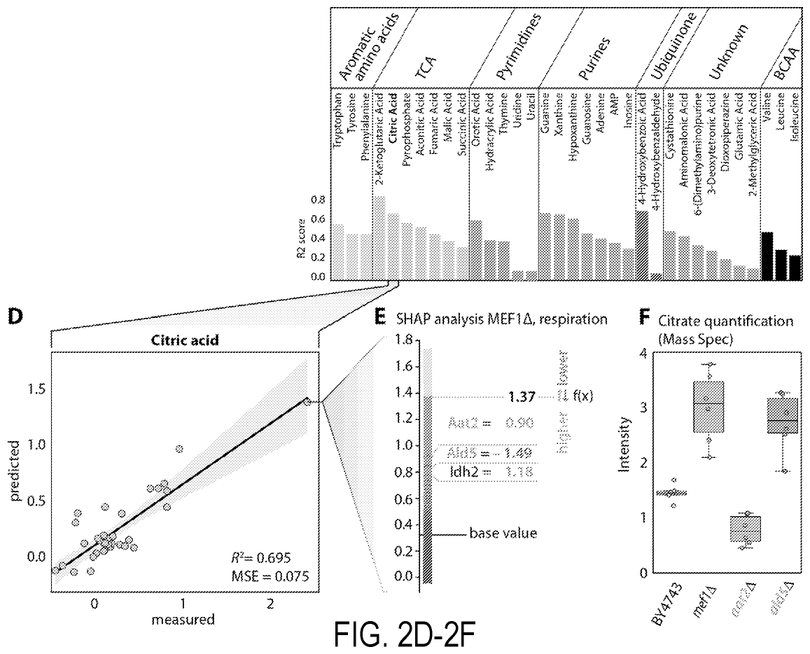

In an example study, the multiomic integration method (“MIM”) described in the present disclosure was evaluated using a tree-based regression model trained to predict metabolite changes from proteomic changes (FIG. 2A). It is an aspect of the disclosed systems and methods to determine new connections between proteins and metabolites using SHAP, a machine learning model interpretation method. New connections from SHAP were experimentally verified to represent the amount of control a protein's quantity exerts over a given metabolite. Many of these protein-metabolite connections are distant based on known genetic and metabolic interactions. Finally, summarizing the strength of these protein control values across all metabolites reveals new connections between experimental conditions. In this case where conditions are single gene knockouts, this clustering reveals new functions of both characterized and uncharacterized mitochondrial proteins.

Data were obtained from a previous multiomic study in yeast, which includes the proteome and metabolome of wild-type or one of 174 single gene knockout yeast strains grown under fermentation and respiration conditions, for a total of 348 multiomic profiles after computing change relative to wild-type controls. In total, the overall dataset included 3,690 proteins and 273 metabolites. After imputation, data were split into training (n=313), and test (n=35) datasets. Multiple different models for each metabolite were explored (FIG. 2B) and their performance was determined by mean squared error and R² between test data model predictions and true values. The Extra Trees model was chosen as it had among the best average performance across metabolites (FIG. 2B) and decision tree based models have specialized model interpretation methods. Positive R² scores between true and predicted quantities of metabolites in the test set were observed for nearly all identified metabolites (FIG. 2C).

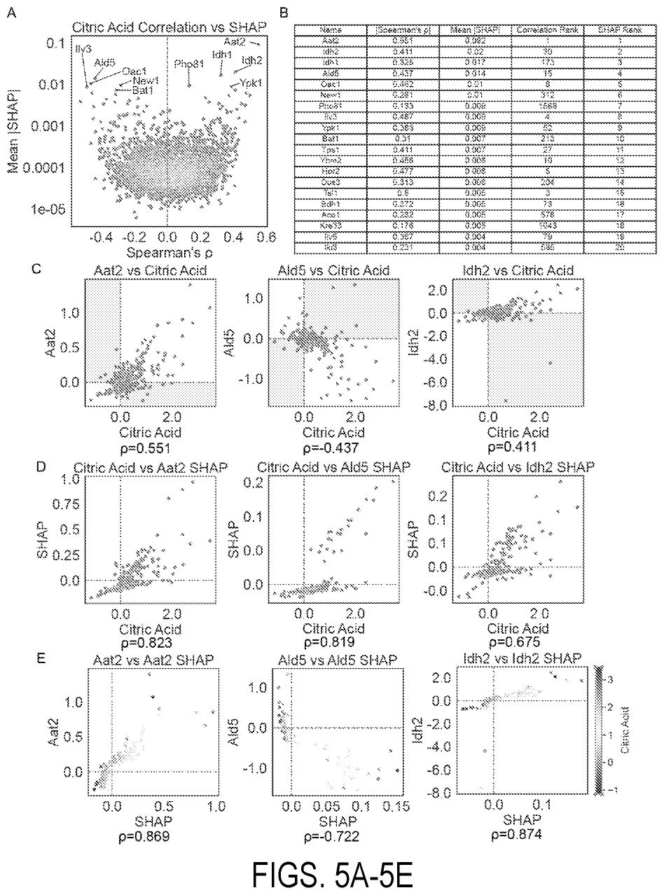

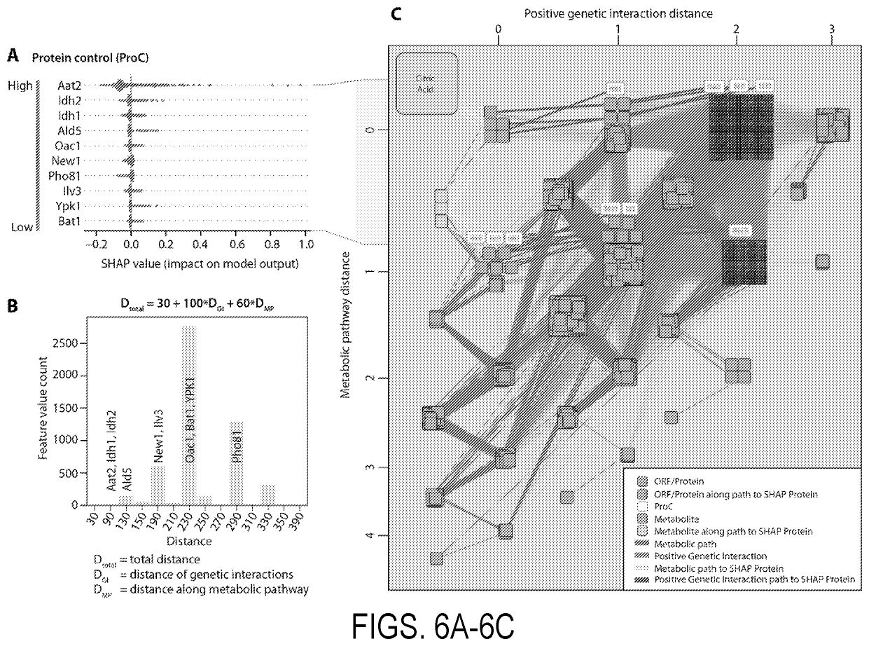

To determine the learned relationships between the proteome and metabolites, TreeSHAP was used to calculate the contribution of each protein input to the predicted level of each of the metabolites across the entire dataset. One well predicted metabolite, citric acid (R²=0.695) was chosen as an example (FIG. 2D, 2E). The proteins with the greatest SHAP value magnitude for MEF1Δ under respiration were AAT2 (25.46% of total magnitude) and ALD5 (4.19%) and IDH2 (3.96%) (FIG. 2F). Unlike previous works that directly measure metabolite-protein interactions, the disclosed systems and methods do not seek to infer the nature of the interaction. Rather, the disclosed systems and methods determine whether specific connections reflect metabolic control by proteins by quantifying metabolites (e.g., citrate in this example) in single gene knockout strains. In the illustrated example, citrate production in AAT2 and ALD5 homozygous deletion mutants were compared to the BY4743 wild-type and a MEF1 deletion mutant (FIG. 2F) and significantly different levels of production were seen between wild type and AAT24 (Student's T-test p-value-7.22E-4), and wild type and ALD5Δ (Student's T-test p-value=1.53E-3), matching the relationships predicted by the SHAP values. This result demonstrates that SHAP values from model interpretation can reveal protein control (“ProC”) over a metabolite to a greater degree than correlations (FIG. 5).

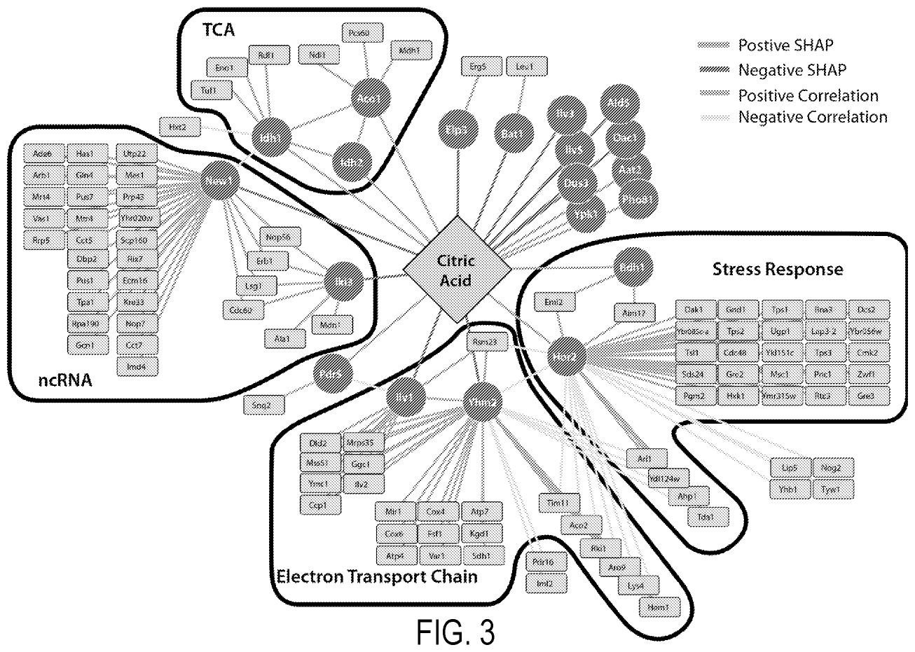

To further explore the relationship between proteins with the highest average ProC over citrate, GO term enrichment was performed (FIG. 3). This analysis revealed several functional pathways that predict citrate related to TCA cycle, stress responses, and respiration, providing further validation that these connections are biologically valid. This may also reflect the logic of the machine learning algorithm and SHAP, choosing as ProCs proteins that are most reflective of these functional pathways and their correlated proteins.

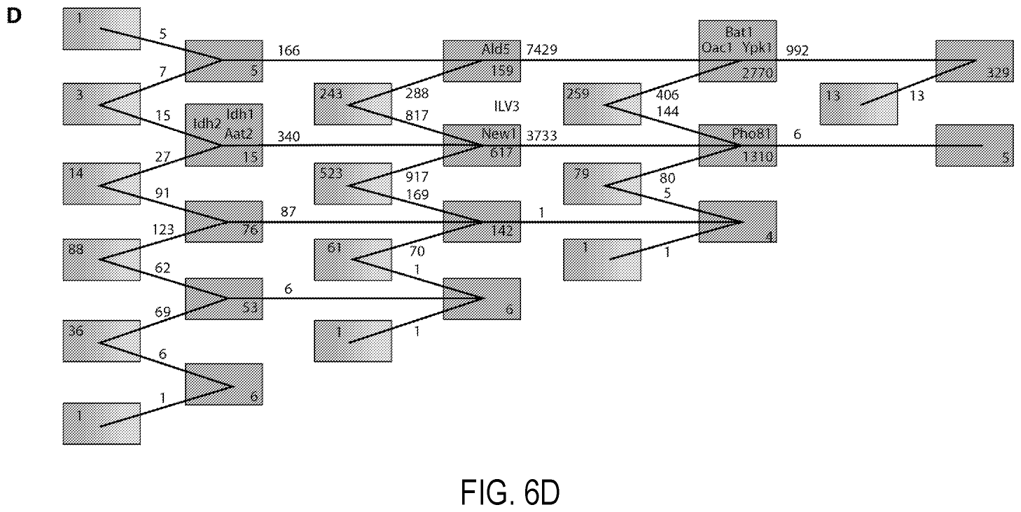

Given that the systems and methods described in the present disclosure are capable of discovering hundreds of new connections between proteins and metabolites, in an example study the discovered connections were evaluated to determine whether they were previously known. The top discovered connections for citrate (FIG. 6A) were mapped onto known positive genetic and metabolic interaction networks (FIG. 6B). AAT2, IDH1, IDH2, and ALD5 were close to citrate, being either one metabolic step, or one positive genetic interaction distance from an enzyme that acts directly on citrate. The remaining connections were more distant, representing new protein connections to citric acid. Notably, OAC1, BAT, YPK1, and PHO81 all lay at the median or above in calculated distance across all proteins and metabolites (FIG. 6C).

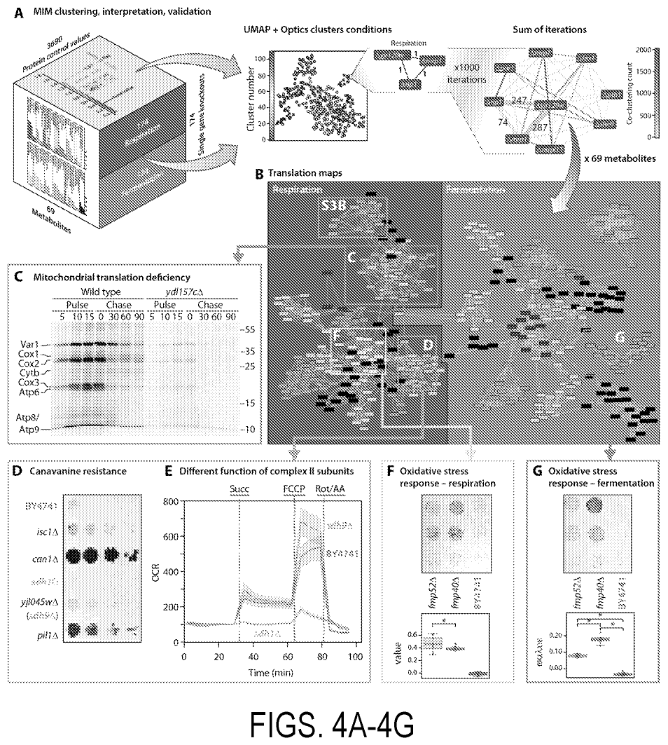

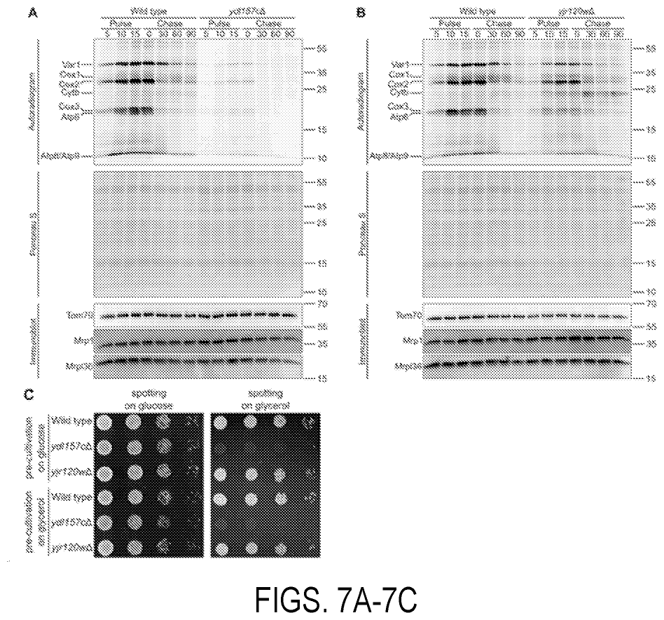

Dimension reduction and clustering of ProC can reveal similarities between the input samples that are not apparent from the omic profiles alone. Because the data used in the aforementioned example study are from single gene knockouts including uncharacterized genes, the experiment tried to predict functions of the genes based on similar ProC profiles. YDL157C and YJR120W are two genes of unknown function associated with the mitochondria. Clustering of knockouts across metabolites (FIGS. 4A and 4B) revealed that these two knockouts frequently cluster with gene knockout strains related to mitochondrial translation. In vivo pulse-chase radiolabeling of mitochondrial translation in wild type and YDL157CΔ and YJR120WΔ revealed changes in mitochondrial translation (FIG. 4C, FIG. 7A, FIG. 7B). YDL157CΔ resulted in a global reduction of mitochondrial translation and YJR120WΔ resulted in a dysregulation of translation. In YJR120WΔ, Var1, Cox2, Cox3, and Atp6 are down regulated, with more extreme downregulation seen in Cox3 and Atp6. Cytb however is upregulated. This alteration in translation reflects known interactions in YJR120W. YJR120W is upstream of ATP2 on the yeast chromosome, and the deletion of YJR120WΔ has been previously noted to alter ATP2's expression. ATP2 is a part of the F1 sector of the F1F10 ATP synthase, which regulates the mitochondrial translation of ATP6 and ATP8. In line with these observations, deletion of YDL157cΔ significantly impaired respiratory growth while the effect of the deletion of YJR120w was less apparent (FIG. 7C).

It is contemplated that the disclosed summary strategy of ProC values can reveal new gene connections that would not be apparent from omic profile similarity alone. To further test the relationships predicted by the clustering network, three additional clusters were analyzed for their connections to incompletely characterized genes. The first of these clusters included YJL045WΔ, now annotated as SDH9 as it is a paralog of SDH1. SDH9Δ was found to have no direct connections to SDH1Δ under respiration conditions in the final trimmed network, but had the greatest connection to PIL1, a key protein in eisosomal structure. The eisosome is a membrane structure involved in membrane transport. One transporter associated with the eisosome is CAN1, an arginine transporter whose deletion confers resistance to the toxic, non-proteinogenic amino acid canavanine. Disruption of the eisosome through deletion of PIL1 has also been shown to provide resistance to canavanine.

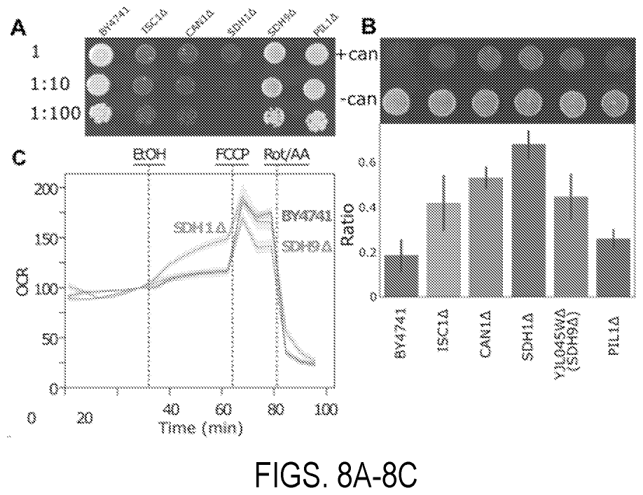

To test the connection between SDH9 and the eisosome, the growth of deletion strains of SDH9, SDH1, CAN1, PIL1, and another connection to PIL1, ISC1, were tested on synthetic complete media (SC) without arginine+canavanine. All tested strains, other than SDH1Δ, which had a growth defect on SC−arg (FIG. 8A), were shown to grow in the presence of canavanine better than wild type (FIG. 4D). Additionally, all strains but PIL1Δ showed significantly higher viability when exposed to very high concentrations of canavanine over 72 hours (FIG. 8B). However, as SDH1Δ showed a growth defect on SC−arg, the link between SDH1 and eisosomal function remains ambiguous.

To test the link between SDH1 and SDH9, respiratory responses were quantified; succinate was used as a source of electrons to complex II and SDH9Δ showed a response more similar to wild type than SDH1Δ. Oxygen consumption rate (OCR) spiked in SDH9Δ when exposed to succinate, while this was not observed in SDH1Δ. (FIG. 4E). The different responses to succinate demonstrate the distinctiveness of the two succinate dehydrogenases and suggest unique functions for each.

Also of note is the resistance of ISC1Δ to canavanine. ISC1 is an enzyme involved in sphingolipid hydrolysis to ceramides and is activated by cardiolipin. Proteins involved in cardiolipin biosynthesis are significantly enriched in the cluster containing ISC1Δ and PIL1Δ. This supports an interplay between cardiolipin, ceramides, and the eisosome.

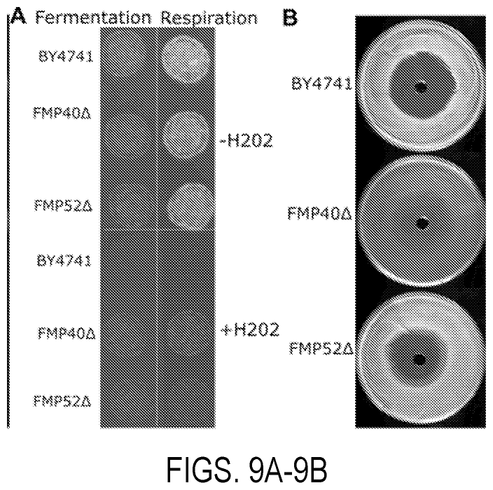

The final two clusters analyzed include another uncharacterized gene in both respiration and fermentation conditions, FMP52Δ. FMP52Δ was found to have the greatest connection weight to FMP40Δ. FMP40 is an AMPylator involved in the oxidative stress response. In addition, FMP52 had the second greatest connection weight to AIM25, a protein of unknown function involved in the oxidative stress response. Based on these connections, it seemed likely that FMP52Δ would have an altered response to oxidative stress and therefore show a difference in resistance to oxidative stressors, such as hydrogen peroxide. To test this hypothesis, cells under respiration and fermentation conditions were exposed to hydrogen peroxide and their viability was determined after 30 minutes (FIG. 4F, FIG. 9A). The resistance to hydrogen peroxide was significantly higher in both FMP40 and FMP52 deletion strains compared to WT controls. Under fermentation conditions, there was a significant difference between the resistance of FMP40Δ and FMP52Δ, while under respiration conditions there was no significant difference. This coincides with the weight of the connections between FMP40 and FMP52 in the network; the weight of the edge connecting them is substantially larger in the respiration cluster. As a separate test, FMP40Δ and FMP52Δ were grown under respiration conditions in a zone of inhibition assay with hydrogen peroxide. A similar result was found, with both the FMP40 and FMP52 lawns growing closer to the source of hydrogen peroxide (FIG. 9B).

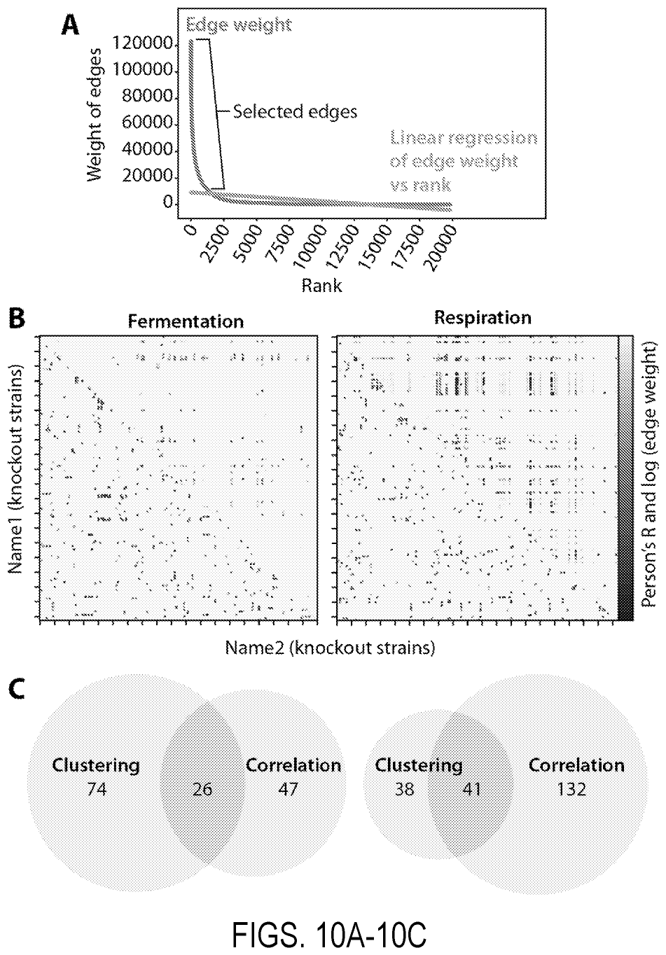

To compare the performance of this clustering method with proteomic correlations, the representation of known genetic and physical interactions among the top selected connections from the clustering analysis and the correlations between proteomes of knockout strains were analyzed. As an example, of the 873 known genetic and physical interactions between the genes represented by the knockout strains under fermentation conditions, 45 were uniquely represented across all proteomic correlations, 31 shared by correlations and clustering, and 85 uniquely represented by clustering analysis (FIG. 10).

These example studies demonstrate that SHAP model explanation values can reflect true biological relationships between the proteome and metabolome (or other omic layers), demonstrated the application of SHAP model explanation values in the integration of multiomic data, and illustrated the utility of this framework through the characterization of several uncharacterized yeast genes. The disclosed systems and methods can be advantageous for multiomic integration and that provide unique insight into the relationships between different multiomic levels.

In the foregoing example study, the following methods were implemented.

A total of 873 proteins were measured in all samples. Missing protein values were imputed using the sklearn function KNNImputer with setting n_neighbors=2, resulting in all 3,690 protein quantities being used as input for the modeling task. Metabolite data were imputed using the same setting, producing 273 complete metabolite columns.

The data were split into 313 random examples for training and 35 examples for testing. This split ratio of 90/10 was chosen arbitrarily based on the ability to have over 300 training examples to learn from while still having a good number of 35 held-out test examples. In other examples, a different split ratio may be used when training a machine learning model according to the examples described in the present disclosure. Multiple types of models were first tested by 5-fold cross validation with the default parameters, and the average mean squared error (MSE) across the five folds were compared. Tested models were implemented in sklearn including: a dummyRegressor baseline, LinearRegression, Lasso, ElasticNet, Ridge, support vector regression wrapped in MultiOutputRegressor, AdaBoost wrapped in MultiOutputRegressor with 500 estimators, GradientBoostingRegressor with 500 estimators wrapped in MultiOutputRegressor, ExtraTreesRegressor with 500 estimators, and RandomForestRegressor with 500 estimators. All of these models except the dummy, ElasticNet, and Lasso performed similarly according to the metric MSE; ExtraTreesRegressor was selected to provide the interpretability of a tree model and the speed of training ExtraTrees.

One multi-output regression Extra Trees model was optimized using 5-fold cross validation with the 313 training examples by grid-search with the following parameters: ‘max_depth’: [10, 30, 50, 70, None], ‘min_samples_leaf’: [1, 2, 5], ‘min_samples_split’: [2, 5, 10], ‘max features’: [‘log 2’, ‘auto’m ‘sqrt’], ‘n_estimators’: [500, 1000, 1500].

The best model parameters for the polar metabolomics model used all of the default parameters except: max_depth=50, n_estimators=500. Those parameters were then used to train a single output ExtraTrees model for each of the 273 polar metabolites. The trained model was used to make predictions on the 35 examples in the test set, and those true and predicted values were used to compute regression metrics. The R2_score and mean_square_error functions in sklearn summarized performance across all the metabolites.

SHAP values were calculated for each knockout for each metabolite model using the TreeExplainer method in the python package SHAP. Only identified metabolites that had a positive R2 score comparing the true versus predicted quantity were included in subsequent analysis. This excludes roughly 200 additional unidentified metabolites.

Correlations between each protein quantity across all single knockout samples were calculated using Spearman's rho and significance was adjusted using Bonferroni Correction. For citric acid, the top 20 mean magnitude SHAP contributor proteins were chosen for further analysis. A network was created with citric acid as the central node, linked to each SHAP contributor protein. Each SHAP contributor protein was then linked to each correlated protein where, correlations between correlated proteins were defined as Bonferroni adjusted P-value <0.05 and a q>0.7 from Spearman rank correlation analysis. Enrichment analysis was performed using ClueGO on each group of SHAP contributor proteins sharing positive correlations and their positively correlated proteins compared against the set of proteins quantified. Significance for terms was determined by Fisher's exact test with Benjamini-Hochberg correction for multiple hypothesis testing.

Yeast strains were grown overnight in YPD at 30° C. After growth, OD595 was measured and cells were washed with PBS. YPDG was inoculated to an initial OD595 of 0.01 and grown at 30° C. for 24 hours. After growth, OD595 was measured and the equivalent of 0.37 OD595 at 1 ml was harvested from each. These cells were pelleted, washed with PBS, pelleted, frozen with LN2, and stored at −80° C. To extract metabolites, each pellet was resuspended in 185 μl 75% methanol, placed at 100° C. for 5 minutes, vortexed for 30 seconds, and cooled on ice. Cell debris was pelleted and the supernatant was used for citrate quantification.

Mass spectrometry was performed on a Thermo Scientific Exploris 240, using a Thermo Scientific Nanospray Ion Source. One ul of each extract was directly infused into the mass spectrometer. To quantify citrate, targeted MS/MS was performed, targeting the ion at 191.0192 m/z. The measured intensity of the fragment at 111.008 m/z was integrated across 811 scans to determine the total citrate present in each sample. Data analysis was performed using pyteomics.

SHAP values of the knockouts were clustered using a combination of Uniform Manifold Approximation and Projection (UMAP) and Ordering Points To Identify Cluster Structure (OPTICS) to determine clustering and likely function of unknown mitochondrial genes. For UMAP, the dimensionality of data (n_components) was set at 10, neighbors (n_neighbors) was set to 3, minimum distance (min_dist) was set to 0, and the distance metric (metric) was manhattan. For OPTICS, the minimum number of samples (min_samples) was set to 2. All other parameters were set to their defaults.

To generate the final clusters and account for the stochasticity of UMAP, UMAP and OPTICS clustering was repeated 1000 times for each metabolite. The clusters generated from each repetition were compared by creating a network with each node representing one of the knockouts and each weighted edge representing twice the number of times the knockouts clustered together of the 1000 repetitions.

The weighted edges, representing the membership of clusters, were combined across known, non-repeated metabolites with a model performance of R²>0. To determine a subset of the most relevant connections, a linear regression was calculated between the edge weight and the rank of the edge when sorted in descending order. All edges with a weight that lay above the linear regression (a weight of 8210) were included as the relevant connections. Nodes were clustered in Cytoscape using the Markov Cluster Algorithm (MCL Cluster in clusterMaker). Layout of the network was calculated using the Prefuse Force Directed Layout.

To create the yeast metabolic network, a list of reactions, enzymes, compounds, and enzymatic reactions was downloaded from Reactome. These datasets were combined to create a metabolic network consisting of all known pathways and their associated enzymes. The following nodes and associated edges were removed from the network due to their ambiguity and relative abundance across reactions: “PROTON”, “WATER”, “ATP”, “ADP”, “PPI”, “Pi”, “Protein-L-serine-or-L-threonine”, “Protein-Ser-or-Thr-phosphate”, “AMP”, “NAD”, “NADH”, “CO-A”, “NADP”, “NADPH”, “CARBON-DIOXIDE”, “GLT”, “S-ADENOSYLMETHIONINE”, “OXYGEN-MOLECULE”, “ACETYL-COA”, “AMMONIUM”, “ADENOSYL-HOMO-CYS”, “Nucleoside-Triphosphates”, “Peptides-holder”, “RNA-Holder”, “Cytochromes-C-Oxidized”, “Cytochromes-C-Reduced”, “GDP”, “Ubiquitin-C-Terminal-Glycine”, and “General-Protein-Substrates”. Edges between enzymes and compounds were assigned a weight of 3.

A list of all known Saccharomyces cerevisiae positive genetic interactions was downloaded from the Saccharomyces Genome Database (SGD). Every ORF absent from the network, i.e. those whose protein does not catalyze a metabolic reaction, were added as nodes and edges with a weight of 10 were created to link ORF nodes with known positive interactions. Weighted closest distance to citrate was calculated for every node using Dijkstra's algorithm. The closest distance can be summarized as 3+6*(metabolic distance)+10*(positive interaction distance)

A list of all possible pairwise combinations of the 174 proteins represented by the knockout strains was generated. A set of all known genetic and physical interactions for the 174 genes were downloaded from the SGD. For each pairwise combination, it was determined if the pair was correlated through proteomic data, connected through clustering analysis, and if it had known genetic or physical interactions. The overlap of correlations and clustering connections with known interactions was determined and plotted using matplotlib-venn.

All strains used for translation assays were isogenic to Saccharomyces cerevisiae W303 MAT a {leu2-3, 112 trp1-1 can1-100 ura3-1 ade2-1 his3-11, 15} obtained from Euroscarf. Chromosomal modifications were made by PCR-based amplification of cassettes followed by integration via homologous recombination and applying lithium acetate transformation. Transformants were validated via growth on selection media and PCR-based confirmation of locus-specific integration.

Strains for the other assays were in BY4743 background for the citrate quantification or BY4741 for the canavanine and hydrogen peroxide assays. All strains were obtained from Horizon Discovery.

Strains were cultivated at 30° C. and 170 rpm shaking. Full media (YEP) contained 1% yeast extract (Bacto, BD Biosciences), 2% peptone (Bacto, BD Biosciences) and 2% glucose, 2% galactose or 2% glycerol as carbon source. Synthetic complete (SC) media consisted of 0.17% yeast nitrogen base (Difco, BD Bioscience). 0.5% (NH₄)₂SO₄, 20 mg/l adenine, 20 mg/l uracil, 20 mg/l arginine, 15 mg/l histidine, 30 mg/l leucine, 30 mg/l lysine, 15 mg/l tryptophan, 30 mg/l isoleucine, 20 mg/l methionine, 50 mg/l phenylalanine, 20 mg/l threonine, 20 mg/l tyrosine, 150 mg/l valine and carbon sources as indicated above. All components were separately prepared in distilled water, autoclaved (25 min, 121° C., 210 kPa, except histidine and tryptophan, which were sterile filtered using 0.2 μm filters) and mixed before use. For solid media, 2% agar was admixed.

[³⁵S]-methionine-based in vivo labeling of mitochondrial translation products was performed as follows. Cells were grown in SC medium containing galactose as carbon source (SC-Gal) to mid-logarithmic phase (approximately OD₆₀₀=1.5-2) and washed three times in 5 ml H₂O. Strains were subsequently washed once in 5 ml SC-Gal media without amino acids and a volume corresponding to OD₆₀₀=4 was harvested and resuspended in 1.5 ml SC-Gal media without amino acids. Amino acids were admixed (18 μg of each amino acid, without methionine) and incubated for 10 min at 30° C., 600 rpm shaking. To stop cytosolic translation, cycloheximide was added to a final concentration of 150 μg/mL and incubated for 2.5 min at 30° C., 600 rpm shaking. 3 μl of [³⁵S]-methionine (10 mCi/ml) were added to start the labeling reaction. For pulse-labeling, 200 μl aliquots were harvested after 5, 10 and 15 minutes, mixed with 50 μl of Stop solution (1.85 M NaOH; 1 M β-mercaptoethanol; 20 mM PMSF) and 10 μl of 200 mM cold methionine, and placed on ice. To follow stability of newly synthesized mitochondrial proteins, 40 μl of 200 mM cold methionine was added to the remaining cell suspensions and incubated at 37° C., 600 rpm (chase). Thereby, 200 μl samples were harvested 30, 60 and 90 minutes after the addition of cold methionine, mixed with stop solution as described above and placed on ice.

Trichloroacetic acid was added to [³⁵S]-methionine-labeled samples with a final concentration of 14%, incubated for 30 min on ice and subsequently centrifuged for 30 minutes, 20000 g at 4° C. Supernatants were carefully removed and pellets rinsed once in 1 ml 100% acetone. After further centrifugation for 30 min at 20 000 g at 4° C., supernatants were removed and pellets resuspended in 75 μl sample buffer (50 mM Tris-HCl, 2% SDS, 10% glycerol, 0.1% bromophenol blue, 100 mM DTT; adjusted to pH 6.8). Subsequently, samples were incubated for 10 min at 65° C., 1400 rpm shaking. 30 μl of the sample were loaded on 16%/0.2% SDS polyacrylamide/bis-acrylamide gels. After separation, proteins were transferred to a nitrocellulose membrane, which was stained with Ponceau S. Protein standard bands (PageRuler™ Plus Prestained Protein Ladder, ThermoFisher) on the nitrocellulose membrane were marked with diluted [³⁵S]-methionine solution and the membranes were applied for autoradiography. Detection was performed with a Fujifilm FLA-9000 phosphorimager.

Membranes were subsequently applied for immunoblotting, using Mrp1, Mrp136, and Tom 70 specific antibodies, as well as anti-rabbit secondary antibody (Sigma, A0545).

To monitor cellular growth, yeast strains were cultivated in YEP media containing either glucose or glycerol to mid-logarithmic phase (approx. OD₆₀₀ 1.5-2). Cultures were washed three times in YEP media without carbon source and a volume corresponding to OD₆₀₀=1 was harvested. Samples were resuspended in 1 ml YEP media without carbon source and three serial 1:10 dilutions thereof were created. 3 μl of cell suspensions were spotted on YEP agar plates either containing glucose or glycerol as carbon source. Plates were incubated for 2 days at 30° C. and photographed with a VWR GenoPlex system.

Cultures were grown for 18 hours in 1 ml YPD for BY4741 or YPD+G418 for the knockout strains. Cultures were centrifuged at 3000 rcf for 3 minutes and pellets were resuspended in 3 ml YPG. After 24 hours, the cultures were pelleted, washed with SC−Arg+glycerol and adjusted with SC−Arg+glycerol to an OD660 of 0.1 and plated onto SC−Arg+glycerol or SC−Arg+glycerol+canavanine at 0.25 μg/ml plates with dilutions of 1, 1:10, 1:100, 1:1000, 1:2000, 1:4000, 1:8000, and 1:16,000. Plates were incubated at 30° C. and pictures were taken after 1 week and again at 18 days. Images of colony formation were captured using ImageLab software with a Bio-Rad GelDoc.

Cultures were grown for 18 hours in 1 ml YPD or YPD+G418. Cultures were centrifuged at 3000 rcf for 3 minutes and pellets were resuspended in 3 ml YPG. After 24 hours, the cultures were pelleted, washed with SC−Arg+glycerol and adjusted with SC−Arg+glycerol to an OD660 of 0.2. 100 μl was adjusted with SC−Arg+glycerol to an OD660 of 0.1 and plated onto YPD plates with dilutions of 1, 1:10, and 1:100 and refrigerated at 3° C. for 72 hours. The remaining culture was adjusted to an OD660 of 0.1 with SC−Arg+glycerol+1200 μg/ml canavanine (final concentration 600 μg/ml) and incubated with shaking at 30° C. for 72 hours. Cultures OD660 were centrifuged, washed, and adjusted to 0.1 OD with SC−Arg+glycerol. Cultures were then plated onto the previously refrigerated YPD plates at dilutions of 1, 1:10, and 1:100. Plates were incubated at 30° C. for 18 hours. Images of colony formation were captured using ImageLab software with a Bio-Rad GelDoc.

Cultures were grown for 18 hours in 2 ml YPD or YPD+G418. 1 ml of each culture was centrifuged at 3000 ref for 3 minutes and pellets were resuspended in 3 ml YPG and incubated for 24 hours at 30° C. To the remaining preculture, 2 ml YPD was added and incubated at 30° C. for 5 hours. For each set of cultures after incubation, the cultures were pelleted, washed with YPD or YPG and adjusted with YPD or YPG to an OD660 of 0.2. For fermentation, 100 μl of each culture was added to 100 μl YPD or YPD+128 mM hydrogen peroxide. For respiration, 100 μl of each culture was added to 100 μl YPG or YPG+1024 mM hydrogen peroxide. Cultures were exposed to hydrogen peroxide for 30 minutes. After treatment, cells were plated onto YPD plates at dilutions of 1, 1:10, 1:100, and 1:1000. Plates were incubated for 18 h at 30° C. Images of colony formation were captured using ImageLab software using a bio-rad GelDoc.

To quantify the growth of the drop dilution assays, images were exported in the TIF format at a DPI of 600. ImageJ was used to measure the brightness (R+G+B)/3 of circles 0.015 in\hat{ }2. Six circles were used as a background and circles measuring the drops were centered. Circles were drawn after each measurement to mark each location. To calculate growth ratios, the average background measurement was subtracted from each brightness measurement. Then the experimental brightness was divided by the control brightness for each strain to calculate a ratio of growth. Average ratios were plotted in seaborn and differences between strains were compared using ANOVA and Tukey's Post Hoc test.

Cultures were grown for 18 hours in 1 ml YPD or YPD+G418. 1 ml of each culture was centrifuged at 3000 ref for 3 minutes and pellets were resuspended in 3 ml YPG and incubated for 24 hours at 30° C. The OD660 was adjusted to 1 for each culture. 1 ml of culture was plated onto 25 ml YPG plates and allowed to dry. To create the hydrogen peroxide gradient, a central section of each plate was removed using a 1 ml pipette tip. 100 μl 3% hydrogen peroxide was added to the central hole and allowed to diffuse. Plates were incubated for 1 week at 30° C. Images of lawn formation were captured using ImageLab software using a bio-rad GelDoc.

To prepare the seahorse plate, 50 μl of poly-L-lysine (0.1 mg/ml) was added to each well and allowed to sit for 2 hours. The solution was aspirated and washed with 100 μl sterile water. The coated plate was stored at 3° C. until ready for the assay. On the day of the assay, the plate was brought to room temperature and 80 μl of seahorse media was added to each well. An additional 100 μl of seahorse media were added to wells acting as baselines. Injections were prepared to have a final concentration of 5 mM ethanol or succinate, 1 μM FCCP, 1 μM rotenone, and 1 μM antimycin A.

To prepare cells for the seahorse assay, cells were grown overnight in 1 mL YPD. After growth to the stationary phase, cells were pelleted and resuspended in 4 ml YPG. Cells were grown for 25 hours. Cells were pelleted and resuspended in seahorse media (6.6 g/l YNB+NH4SO4) to a final OD660 of 0.38. Each sample was diluted an additional 1:5 in seahorse media and 100 μl of culture were placed into each well of the prepared seahorse plate. The plate was centrifuged at 250 rcf for 3 minutes and incubated at 30° C. for 30 minutes.

Plates were measured on a Seahorse XF-96. A total of 18 measurements of the oxygen consumption rate (OCR) and extracellular acidification rate (ECAR) were taken over 96 minutes, with 10 technical replicates for each strain. Six initial measurements were taken as a baseline, six measurements were taken after the injection of ethanol or succinate, three measurements after the injection of FCCP, and three final measurements after the injection of rotenone/antimycin A. Data collected were analyzed using Agilent Wave and pandas and plotted using seaborn and matplotlib.

As described above, in an example study data were obtained from a previous multiomic study in yeast containing the proteome and metabolome of wild-type or one of 174 single gene knockout yeast strains grown under fermentation and respiration conditions, for a total of 348 multiomic profiles after computing change relative to wild-type controls. In total, the overall dataset included 3690 proteins and 273 metabolites. After imputation, data were split into training (n=313) and test (n=35) datasets. Multiple different models for each metabolite were explored (FIG. 2B), and their performance was determined by mean squared error and R² between test data model predictions and true values. The Extra Trees model was chosen as it had among the best average performance across metabolites (FIG. 2B) and decision tree-based models have specialized model interpretation methods. Positive R² scores between true and predicted quantities of metabolites in the test set were observed for nearly all identified metabolites (FIG. 2C).

To determine the learned relationships between the proteome and metabolites, TreeSHAP was used to calculate the contribution of each protein input to the predicted level of each of the metabolites across the entire dataset. One well-predicted metabolite, citric acid (R²=0.695) was chosen as an example (FIGS. 2D and 2E). Citrate was chosen because it is extensively studied as part of the TCA cycle, and as an abundant and ionizable metabolite, perturbations to citrate's quantity can be easily measured by mass spectrometry. The proteins with the greatest SHAP value magnitude for mef1Δ under respiration were Aat2 (25.46% of total magnitude), Ald5 (4.19%), and Idh2 (3.96%) (FIG. 2F). This suggests that in the MEF1 knockout, the resulting changes in these three proteins are driving the difference in citrate, not the absence of Mef1 protein. Unlike previous works that directly measure metabolite-protein interactions, the nature of the interaction between citrate and these proteins is not inferred. It was instead investigated whether these connections reflect metabolic control by proteins by quantifying citrate in single gene knockout strains. Aat2 and Ald5 proteins were chosen for this follow-up experiment because they were the most important for explaining this gene knockout, and they were among the most important for explaining citrate across all knockouts in the test set. Citrate production in AAT2 and ALD5 homozygous deletion mutants were compared to the BY4743 wild-type and a MEF1 deletion mutant (FIG. 2F) and significantly different citrate abundance was seen between wild-type and aat2Δ (Student's 1-test P-value=7.22E-4) and wild-type and ald5Δ (Student's 1-test P-value=1.53E-3), matching the relationships predicted by the SHAP values. This result suggests that SHAP values from model interpretation may reveal ProC over a metabolite to a greater degree than correlations (FIG. 5).

To further explore the relationship between proteins with the highest average ProC over citrate, GO term enrichment was performed (FIG. 3). This analysis revealed several functional pathways that predict citrate related to the TCA cycle, stress responses, and respiration, providing further validation of these new protein connections to citrate discovered by MIMaL. This may also reflect the logic of the machine learning algorithm and SHAP, choosing proteins most reflective of these functional pathways and their correlated proteins.

Given that the systems and methods described in the present disclosure may discover hundreds of new connections between proteins and metabolites, it was investigated whether these connections are largely new or known. To determine this, the top 10 proteins with the greatest overall average magnitude of SHAP values were analyzed. These top discovered connections for citrate (FIG. 6A) were mapped onto known positive genetic and metabolic interaction networks (FIG. 6B). AAT2, IDH1, IDH2 and ALD5 were close to citrate, being either one metabolic step, or one positive genetic interaction distance from an enzyme that acts directly on citrate. The remaining connections were more distant, representing new protein connections to citric acid. Notably, OAC1, BAT1, YPK1 and PHO81 all lay at the median or above in calculated distance across all proteins and metabolites (FIG. 6C).

Because the data used here are from single gene knockouts including uncharacterized genes, it was investigated where the similarity of ProC profiles could be used to predict gene function. Dimension reduction and clustering of ProC profiles were used for each metabolite to discover relationships between conditions (see FIG. 4).

YDL157C and YJR120W are two genes of unknown function associated with the mitochondria. Clustering of knockouts across metabolites (FIGS. 4A and 4B) revealed that these two knockouts frequently cluster with gene knockout strains related to mitochondrial translation. In vivo pulse-chase, radiolabeling of mitochondrial translation in wild-type and ydl157cΔ and yjr120wΔ revealed changes in mitochondrial translation (FIG. 4C, FIG. 7A, FIG. 7B).

ydl157cΔ had a global reduction of mitochondrial translation, and the absence of YJR120W resulted in a dysregulation of translation. In yjr120wΔ, Var1, Cox2, Cox3 and Atp6 are down regulated, with more pronounced downregulation seen for Cox3 and Atp6. Cytb however was upregulated. This alteration in translation might reflect previously suggested interactions of YJR120W. YJR120W is upstream of ATP2 on the yeast chromosome, and the deletion of YJR120W was previously noted to alter ATP2's expression. Atp2 is a part of the F1 sector of the FIFo ATP synthase, which regulates the mitochondrial translation of ATP6 and ATP8. In line with these observations, the deletion of YDL157C severely impaired respiratory growth, while the effect of the deletion of YJR120W was less pronounced (FIG. 7C). YJR120W and YDL157C may be referred to as ‘Determines Mitochondrial prOteome’ or DMO1 and DM (2, respectively.

Although the connections between translation and YDI157C and YJR120W were not previously discovered, closer inspection of the correlation between proteome profiles resulting from gene knockouts may have revealed this relationship. It is an advantage of the systems and methods described in the present disclosure that the our summary strategy of ProC values can reveal new gene connections that may not be apparent from an omic profile similarity alone.

To further test the relationships predicted by the clustering network, three additional clusters were analyzed for their connections to incompletely characterized genes. The first of these clusters included YJL045W, now annotated as SDH9 as it is a paralog of SDH1. Unexpectedly, sdh9Δ was found to lack direct connections to sdh1Δ under respiration conditions in the final trimmed network, but rather had the greatest connection to Pill, a key protein in eisosomal structure. The eisosome is a membrane structure involved in membrane transport. One transporter associated with the eisosome is Can1, an arginine transporter whose deletion confers resistance to the toxic, non-proteinogenic amino acid canavanine. Disruption of the eisosome through deletion of PIL1 has also been shown to provide resistance to canavanine. To test the connection between SDH9 and the eisosome, the growth of deletion strains of SDH19, SDH1. CAN1, PIL1, and another connection to PIL1, ISC1, were tested on SC media without arginine canavanine. All tested strains, other than sdh1Δ, which had a growth defect on SC−arg (FIG. 8A), were shown to grow in the presence of canavanine better than wild-type (FIG. 3D). Additionally, all strains but pil1Δ showed significantly higher viability when exposed to very high concentrations of canavanine over 72 hours (FIG. 8B).

To test the link between SDH1 and SDH9, respiratory responses were quantified; succinate was used as a source of electrons to complex II and sdh9Δ showed a response more similar to wild-type than sdh1Δ. OCR spiked in sdh9Δ when exposed to succinate, while this was not observed in sdh1Δ (FIG. 4E). The different responses to succinate demonstrate the distinctiveness of the two succinate dehydrogenases and suggest unique functions for each.

Also of note is the resistance of isc1Δ to canavanine. Isc1 is an enzyme involved in sphingolipid hydrolysis to ceramides and is activated by cardiolipin. Proteins involved in cardiolipin biosynthesis are significantly enriched in the cluster containing isc1Δ and pil1Δ. This supports an interplay between cardiolipin, ceramides and the eisosome.

The final two clusters analyzed include another uncharacterized gene in both respiration and fermentation conditions, FMP52. fmp52Δ was found to have the greatest connection weight to fmp40Δ. Fmp40 is an AMPylator involved in the oxidative stress response. In addition, Fmp52 had the second greatest connection weight to Aim25, a protein of unknown function involved in the oxidative stress response.

Based on these connections, it seemed likely that fmp52Δ would have an altered response to oxidative stress and therefore show a difference in resistance to oxidative stressors, such as hydrogen peroxide. To test this hypothesis, cells under respiration and fermentation conditions were exposed to hydrogen peroxide and their viability was determined after 30 minutes (FIGS. 4F and 4G, FIG.). The resistance to hydrogen peroxide was significantly higher in both FMP40 and FMP52 deletion strains compared to WT controls. Under fermentation conditions, there was a significant difference between the resistance of fmp40Δ and fmp52Δ, while under respiration conditions there was no significant difference. This coincides with the weight of the connections between FMP40 and FMP52 in the network; the weight of the edge connecting them is substantially larger in the respiration cluster. As a separate test, fmp40Δ and fmp52Δ were grown under respiration conditions in a zone-of-inhibition assay with hydrogen peroxide. A similar result was found, with both the fmp40Δ and fmp52Δ lawns growing closer to the source of hydrogen peroxide (FIG. 9B).

To compare the performance of this clustering method with proteomic correlations, the representation of known genetic and physical interactions among the top selected connections were analyzed from the clustering analysis and the correlations between proteomes of knockout strains. As an example, of the 873 known genetic and physical interactions between the genes represented by the knockout strains under fermentation conditions, 45 were uniquely represented across all proteomic correlations, 31 shared by correlations and clustering, and 85 uniquely represented by clustering analysis (FIG. 10).

It has been shown that the systems and methods described in the present disclosure provide for machine learning models that can effectively predict one layer of omic data from another layer of omic data. Additionally, it is an advantage of the systems and methods described in the present disclosure that SHAP model interpretation values can reflect true biological relationships that represent ProC over a metabolite. As one example case, MIMaL validated that two proteins predicted to control citrate that are not directly involved in producing or consuming citrate based on known metabolism pathways. More generally, it was found that pathway enrichment analysis of proteins that control citrate reveal expected and new pathways that regulate citrate. Network analysis for all discovered proteins that interact with citrate revealed that most discovered connections are distant based on known genetic and metabolic interactions.

It is an advantage of the disclosed systems and methods that machine learning model interpretation with SHAP can reveal how proteins control metabolites globally. This enables the exploration of the utility of the SHAP-derived ProC values, and demonstrates that ProC values derived from model interpretation can reveal functions for characterized and uncharacterized genes.

As one additional example demonstrating the utility of these predicted ProC values, dimension reduction and clustering of ProC values was used to discover similarity between experimental conditions. When each study condition is a single gene knockout, this analysis may reveal new connections between those genes in the form of a similarity network. The similarities revealed by this method are different from those obtained from simply clustering the proteomics profiles from each condition. The utility of this method was demonstrated by predicting and validating functions for several uncharacterized and characterized yeast genes.

Although the example studies described above focused on proteomic and metabolomic data integration, the systems and methods described in the present disclosure can be used to discover connections between any two omic layers.

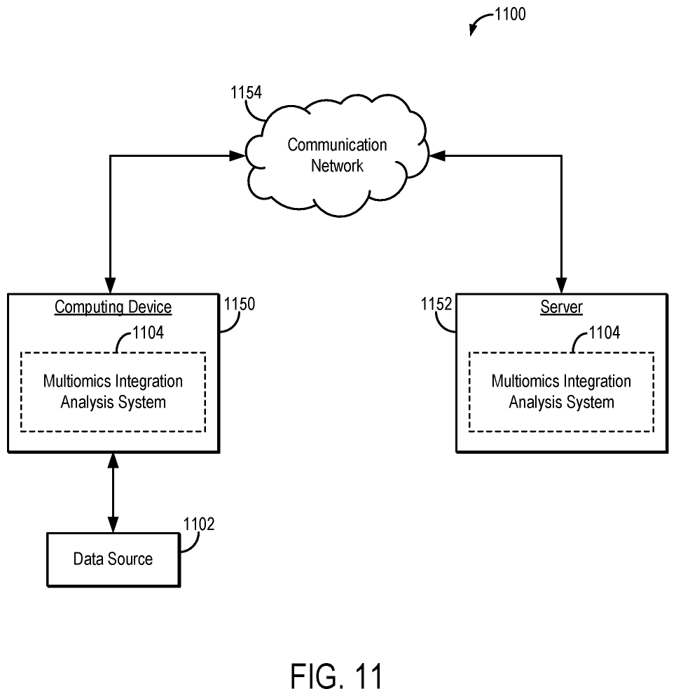

Referring now to FIG. 11, an example of a system 1100 for multiomics integration analysis in accordance with some embodiments of the systems and methods described in the present disclosure is shown. As shown in FIG. 11, a computing device 1150 can receive one or more types of data (e.g., proteomics data, metabolomics data, multiomics datasets) from data source 1102. In some embodiments, computing device 1150 can execute at least a portion of a multiomics integration analysis system 1104 to generate feature data indicative of connections between different layers of an input multiomics dataset received from the data source 1102.

Additionally or alternatively, in some embodiments, the computing device 1150 can communicate information about data received from the data source 1102 to a server 1152 over a communication network 1154, which can execute at least a portion of the multiomics integration analysis system 1104. In such embodiments, the server 1152 can return information to the computing device 1150 (and/or any other suitable computing device) indicative of an output of the multiomics integration analysis system 1104.

In some embodiments, computing device 1150 and/or server 1152 can be any suitable computing device or combination of devices, such as a desktop computer, a laptop computer, a smartphone, a tablet computer, a wearable computer, a server computer, a virtual machine being executed by a physical computing device, and so on.

In some embodiments, data source 1102 can be any suitable source of data (e.g., measurement data, multiomics data), another computing device (e.g., a server storing measurement data, multiomics data), and so on. In some embodiments, data source 1102 can be local to computing device 1150. For example, data source 1102 can be incorporated with computing device 1150 (e.g., computing device 1150 can be configured as part of a device for measuring, recording, estimating, acquiring, or otherwise collecting or storing data). As another example, data source 1102 can be connected to computing device 1150 by a cable, a direct wireless link, and so on. Additionally or alternatively, in some embodiments, data source 1102 can be located locally and/or remotely from computing device 1150, and can communicate data to computing device 1150 (and/or server 1152) via a communication network (e.g., communication network 1154).

In some embodiments, communication network 1154 can be any suitable communication network or combination of communication networks. For example, communication network 1154 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1154 can be a local area network, a wide area network, a public network (e.g., the Internet), a private or semi-private network (e.g., a corporate or university intranet), any other suitable type of network, or any suitable combination of networks. Communications links shown in FIG. 11 can each be any suitable communications link or combination of communications links, such as wired links, fiber optic links, Wi-Fi links, Bluetooth links, cellular links, and so on.

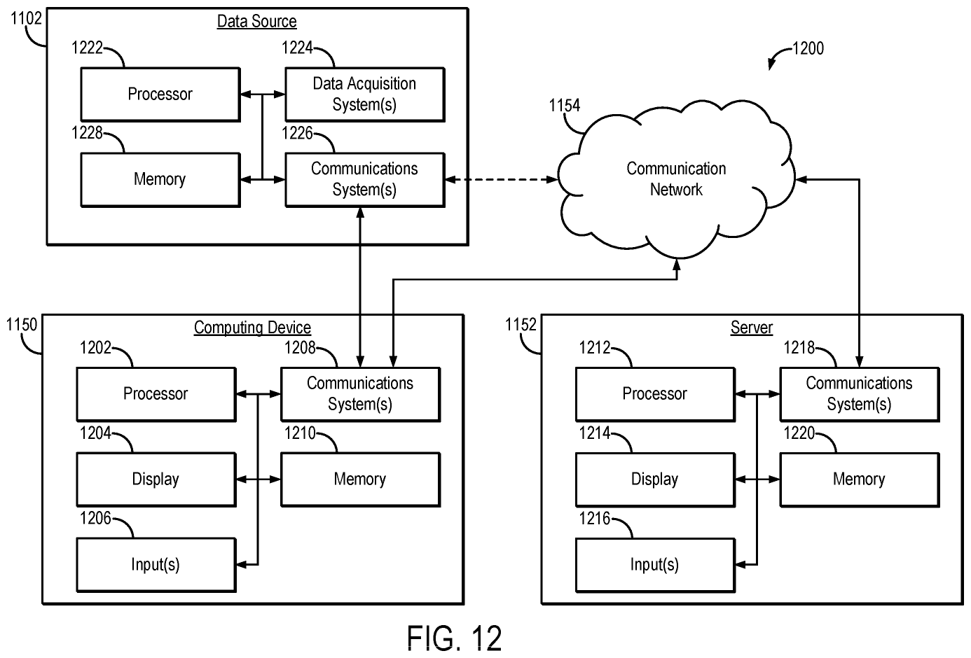

Referring now to FIG. 12, an example of hardware 1200 that can be used to implement data source 1102, computing device 1150, and server 1152 in accordance with some embodiments of the systems and methods described in the present disclosure is shown.

As shown in FIG. 12, in some embodiments, computing device 1150 can include a processor 1202, a display 1204, one or more inputs 1206, one or more communication systems 1208, and/or memory 1210. In some embodiments, processor 1202 can be any suitable hardware processor or combination of processors, such as a central processing unit (“CPU”), a graphics processing unit (“GPL”), and so on. In some embodiments, display 1204 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1206 can include any suitable input devices and/or sensors that can be used to receive user input, such as a keyboard, a mouse, a touchscreen, a microphone, and so on.

In some embodiments, communications systems 1208 can include any suitable hardware, firmware, and/or software for communicating information over communication network 1154 and/or any other suitable communication networks. For example, communications systems 1208 can include one or more transceivers, one or more communication chips and/or chip sets, and so on. In a more particular example, communications systems 1208 can include hardware, firmware, and/or software that can be used to establish a Wi-Fi connection, a Bluetooth connection, a cellular connection, an Ethernet connection, and so on.

In some embodiments, memory 1210 can include any suitable storage device or devices that can be used to store instructions, values, data, or the like, that can be used, for example, by processor 1202 to present content using display 1204, to communicate with server 1152 via communications system(s) 1208, and so on. Memory 1210 can include any suitable volatile memory, non-volatile memory, storage, or any suitable combination thereof. For example, memory 1210 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1210 can have encoded thereon, or otherwise stored therein, a computer program for controlling operation of computing device 1150. In such embodiments, processor 1202 can execute at least a portion of the computer program to present content (e.g., images, user interfaces, graphics, tables), receive content from server 1152, transmit information to server 1152, and so on. For example, the processor 1202 and the memory 1210 can be configured to perform the methods described herein (e.g., the method of FIG. 1).

In some embodiments, server 1152 can include a processor 1212, a display 1214, one or more inputs 1216, one or more communications systems 1218, and/or memory 1220. In some embodiments, processor 1212 can be any suitable hardware processor or combination of processors, such as a CPU, a GPU, and so on. In some embodiments, display 1214 can include any suitable display devices, such as an LCD screen, LED display, OLED display, electrophoretic display, a computer monitor, a touchscreen, a television, and so on. In some embodiments, inputs 1216 can include any suitable input devices and/or sensors that can be used to receive user input, such as a keyboard, a mouse, a touchscreen, a microphone, and so on.

In some embodiments, communications systems 1218 can include any suitable hardware, firmware, and/or software for communicating information over communication network 1154 and/or any other suitable communication networks. For example, communications systems 1218 can include one or more transceivers, one or more communication chips and/or chip sets, and so on. In a more particular example, communications systems 1218 can include hardware, firmware, and/or software that can be used to establish a Wi-Fi connection, a Bluetooth connection, a cellular connection, an Ethernet connection, and so on.

In some embodiments, memory 1220 can include any suitable storage device or devices that can be used to store instructions, values, data, or the like, that can be used, for example, by processor 1212 to present content using display 1214, to communicate with one or more computing devices 1150, and so on. Memory 1220 can include any suitable volatile memory, non-volatile memory, storage, or any suitable combination thereof. For example, memory 1220 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1220 can have encoded thereon a server program for controlling operation of server 1152. In such embodiments, processor 1212 can execute at least a portion of the server program to transmit information and/or content (e.g., data, images, a user interface) to one or more computing devices 1150, receive information and/or content from one or more computing devices 1150, receive instructions from one or more devices (e.g., a personal computer, a laptop computer, a tablet computer, a smartphone), and so on.

In some embodiments, the server 1152 is configured to perform the methods described in the present disclosure. For example, the processor 1212 and memory 1220 can be configured to perform the methods described herein (e.g., the method of FIG. 1).

In some embodiments, data source 1102 can include a processor 1222, one or more data acquisition systems 1224, one or more communications systems 1226, and/or memory 1228. In some embodiments, processor 1222 can be any suitable hardware processor or combination of processors, such as a CPU, a GPU, and so on. In some embodiments, the one or more data acquisition systems 1224 are generally configured to acquire data. Additionally or alternatively, in some embodiments, the one or more data acquisition systems 1224 can include any suitable hardware, firmware, and/or software for coupling to and/or controlling operations of a data acquisition system (e.g., a mass spectrometry system or other system for acquiring multiomics data types). In some embodiments, one or more portions of the data acquisition system(s) 1224 can be removable and/or replaceable.

Note that, although not shown, data source 1102 can include any suitable inputs and/or outputs. For example, data source 1102 can include input devices and/or sensors that can be used to receive user input, such as a keyboard, a mouse, a touchscreen, a microphone, a trackpad, a trackball, and so on. As another example, data source 1102 can include any suitable display devices, such as an LCD screen, an LED display, an OLED display, an electrophoretic display, a computer monitor, a touchscreen, a television, etc., one or more speakers, and so on.

In some embodiments, communications systems 1226 can include any suitable hardware, firmware, and/or software for communicating information to computing device 1150 (and, in some embodiments, over communication network 1154 and/or any other suitable communication networks). For example, communications systems 1226 can include one or more transceivers, one or more communication chips and/or chip sets, and so on. In a more particular example, communications systems 1226 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1228 can include any suitable storage device or devices that can be used to store instructions, values, data, or the like, that can be used, for example, by processor 1222 to control the one or more data acquisition systems 1224, and/or receive data from the one or more data acquisition systems 1224; to generate images from data; present content (e.g., data, images, a user interface) using a display; communicate with one or more computing devices 1150; and so on. Memory 1228 can include any suitable volatile memory, non-volatile memory, storage, or any suitable combination thereof. For example, memory 1228 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1228 can have encoded thereon, or otherwise stored therein, a program for controlling operation of data source 1102. In such embodiments, processor 1222 can execute at least a portion of the program to generate images, transmit information and/or content (e.g., data, images, a user interface) to one or more computing devices 1150, receive information and/or content from one or more computing devices 1150, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

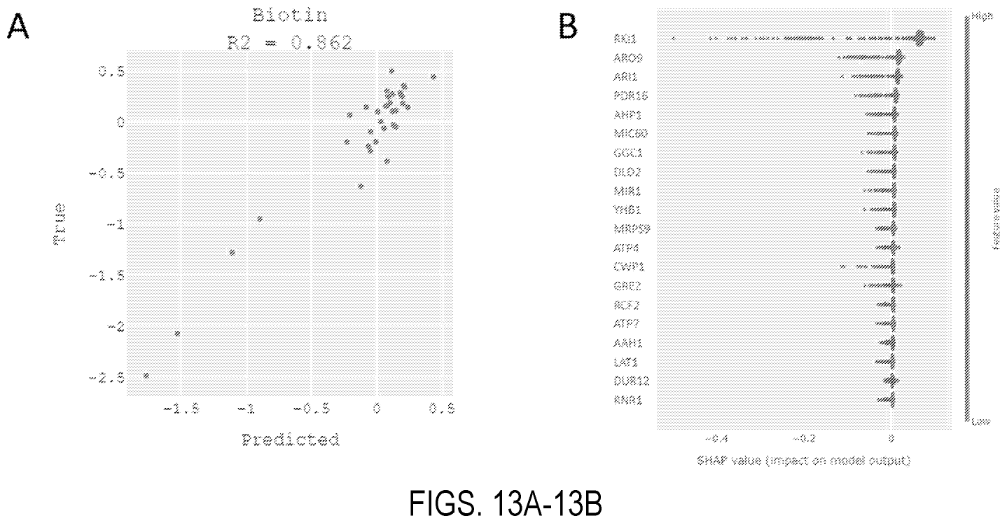

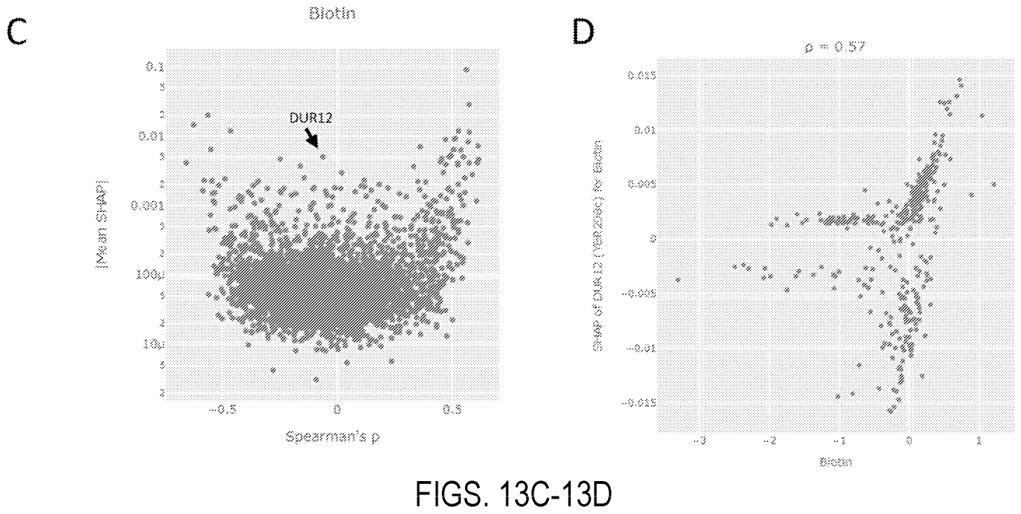

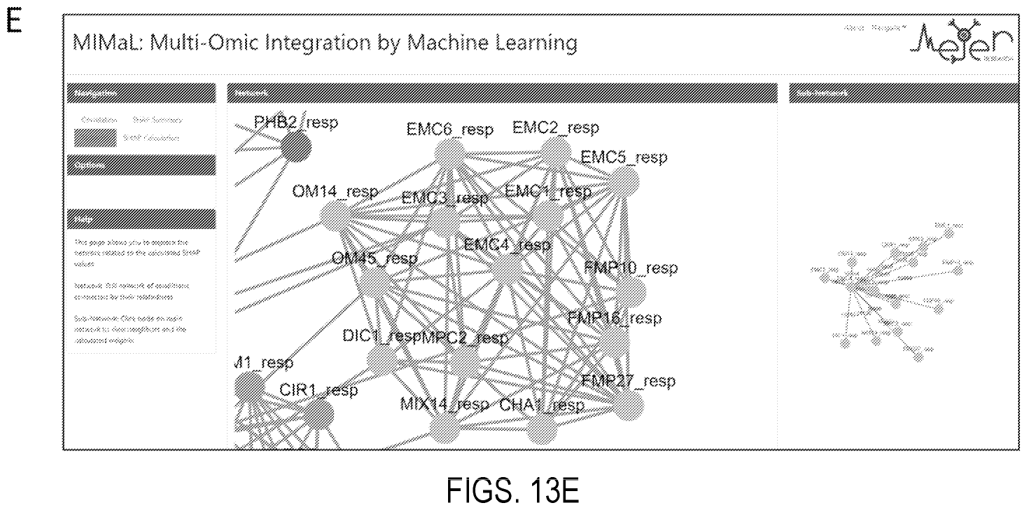

To facilitate exploration of the results and data generated using the systems and methods described in the present disclosure, a graphical user interface can be generated and implemented using, for example, the computing device 1150 and/or server 1152. Example portions of such as graphical user interface are shown in FIGS. 13A-13E. The model performance for any metabolite can easily be checked with a scatterplot on a ‘SHAP Summary’ page; in this case, biotin is shown in FIG. 13A as being well predicted with R² score of 0.862 between true and predicted quantities 13. The 20 most important proteins for predicting biotin are shown in a SHAP summary plot in FIG. 13B, which showed that RKI1 was the most important regulator of biotin's quantity. Comparison of the correlation between every protein's correlation with biotin and the mean average SHAP for that protein showed that some proteins are important for model interpretation (high y value) but have a low correlation with biotin (x value near 0, FIG. 13C). One such example is Dur12 protein. The ‘correlation’ tab allows inspection of the correlation between Dur12 and biotin, which is poor. The correlation between the SHAP value of Dur12's control over biotin versus the quantity of biotin is more correlated, although some interesting patterns in the data are apparent (FIG. 13D).

Using the ‘Network’ tab (FIG. 13E) enables exploration of the network relationships between conditions shown in FIG. 4. For example, if interested in the endoplasmic reticulum membrane complex, the graphical user interface enables a user to zoom in on those points and see that they are connected to several uncharacterized proteins (Fmp10, Fmp16 and Fmp27). They are also connected to some characterized proteins, including Dic1 and Mpc2, which are both transporters of metabolites containing carboxylic acids. A testable hypothesis that users may derive from these data include that ER transmembrane complex proteins may also be involved in mitochondria protein import, or that these carboxylic acid transporters are important for protein folding in the ER.

The graphical user interface also enables MIMaL analysis of arbitrary multiomic datasets uploaded by the user. The input may be multiple molecule measurements from one omic layer and the output may be a single molecule measurement from a different omic layer in the same samples. The systems and methods will train a model and can report the performance via the graphical user interface in the form of the true versus predicted quantities for the output molecule. The graphical user interface may also show the clustered UMAP of the similarity between input conditions based on the SHAP values. An important consideration when using this is that the number of samples should be large, probably at least 100.

The present disclosure has described one or more preferred embodiments, and it should be appreciated that many equivalents, alternatives, variations, and modifications, aside from those expressly stated, are possible and within the scope of the invention.

1. A method for generating feature data indicative of an integration between different layers of multiomics data, the method comprising:
(a) accessing a first omics dataset with a computer system, wherein the first omics dataset comprises a first omics data type;
(b) accessing a machine learning model with the computer system, wherein the machine learning model has been trained on training data to predict a second omics data type from the first omics data type;
(c) inputting the first omics dataset to the machine learning model via the computer system, generating output data as predictive values of a second omics dataset comprising the second omics data type;
(d) generating model interpretation data from at least one of the machine learning model, the first omics dataset, or the output data, wherein the model interpretation data indicate features in the multiomics dataset that are predictive of connections between the first omics dataset and the second omics dataset; and
(e) generating feature data with the computer system based on the model interpretation data, wherein the feature data indicate connections between the first omics dataset and the second omics dataset.

2. The method of claim 1, wherein the feature data indicate predictive connections between the first omics dataset and the second omics dataset.

3. The method of claim 1, wherein the feature data are generated based on a cluster analysis of the model interpretation data.

4. The method of claim 1, wherein the model interpretation data comprise a plurality of features in at least one of the first omics dataset or the second omics dataset.

5. The method of claim 4, wherein the model interpretation data also include rank values of the plurality of features.

6. The method of claim 5, wherein the rank values of the plurality of features indicate a ranking of the plurality of features in terms of relevance to being predictive of connections between the first omics dataset and the second omics dataset.

7. The method of claim 4, wherein the model interpretation data also include quantitative values associated with each of the plurality of features.

8. The method of claim 4, wherein the model interpretation data also include measures of values of the plurality of features having at lease one of a positive predictive effect or a negative predictive effect for being predictive of connections between the first omics dataset and the second omics dataset.

9. The method of claim 1, wherein the model interpretation data comprise shapely additive explanation (SHAP) values.

10. The method of claim 1, wherein the first omics dataset comprises proteomics data.

11. The method of claim 10, wherein the second omics dataset comprises metabolomics data.

12. The method of claim 11, wherein generating the feature data comprises generating protein control (ProC) values from the model interpretation data.

13. The method of claim 12, wherein the ProC values indicate one or more proteins that are predicted to control one or more metabolites.

14. The method of claim 1, comprising performing dimensionality reduction and clustering analysis on the feature data, generating an output that indicates similarities between input conditions associated with at least one of the first omic dataset or the second omics dataset.

15. The method of claim 14, wherein the dimensionality reduction is performed using a uniform manifold projection and approximation.

16. The method of claim 1, wherein the first omics dataset comprises one of proteomics data, metabolomics data, genomics data, epigenomics data, transcriptomics data, or lipidomics data.

17. The method of claim 1, wherein the feature data indicate connections between input conditions comprising single gene knockouts.

18. The method of claim 17, wherein the feature data indicate gene function based on the single gene knockouts.

19. The method of claim 1, wherein the machine learning model comprises a tree-based regression model.

20. The method of claim 19, wherein the tree-based regression model comprises an extremely randomized trees (Extra Trees) model.
